(19) United States
(12) Patent Application Publication
(10) Pub. No.: US 2026/0317260 A1
(43) Pub. Date: Oct. 15, 2026

(54) PROCESS FOR THE PREPARATION OF 1,1,4,4-TETRAALKYLOXY-2-BUTENE

(71) Applicant: DSM IP Assets B.V., Maastricht (NL)

(72) Inventors: Werner BONRATH, Kaiseraugst (CH); Roman GOY, Kaiseraugst (CH); Marcel JORAY, Kaiseraugst (CH); Ralph WAECHTER, Kaiseraugst (CH)

(21) Appl. No.: 19/479,909

(22) PCT Filed: May 3, 2024
(86) PCT No.: PCT/EP2024/062222

§ 371 Date: Oct. 29, 2025

(30) Foreign Application Priority Data
May 5, 2023 (EP) 23171762.0

(51) Int. Cl. C07C 41/50

(52) U.S. Cl. CPC C07C 41/50

(57) ABSTRACT

The present invention relates to a novel process for the preparation of alkoxylated 2,5-dihydrofuran.

The present invention relates to a novel process for the preparation of 1,1,4,4-tetraalkyloxy-2-butene.
1,1,4,4-tetraalkyloxy-2-butene, which are the compounds of formula (I)

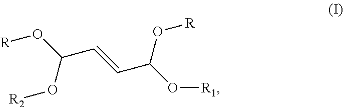

wherein R, R₁ and R₂ are independently from each other linear or branched C₁-C₆ alkyl group, are very useful compounds.

For example, 1,1,4,4-tetramethoxy-2-butene (wherein R, R₁ and R₂ are all —CH₃) is an important intermediate in the synthesis of carotenoids (known from prior art, i.e. from CN 108 752 178 A).

The prior art discloses various methods to produce 1,1,4,4-tetraalkyloxy-2-butene. One of the syntheses is shown in the following reaction scheme:

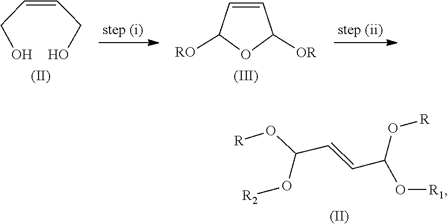

wherein (2Z)-but-2-ene-1,4-diol (compound of formula (II)) is alkylated in a first step (step (i)), wherein 2,5-dihydro-2,5-dialkoxyfuran is obtained and then in a second step (step (ii)) the desired product (compound of formula (I)) is obtained.

Due to the importance of the compounds of formula (I), there is always a need for an improved synthesis for these compounds.

Surprisingly, it was found when the process was carried out under specific conditions, the compounds of formula (I) are obtained in excellent yield and the waste products of the new process are kept at a very low level.

As stated above, the process according to the present invention consists of two steps (step (i)) and step (ii)).

In the following the steps are discussed in more details.

### Step (i)

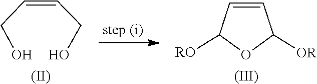

This step is carried out electrochemically. Step (i) can be carried out as described i.e. in WO2006/100289, wherein 2,5-dihydrofuran derivatives (compounds of formula (III)) by electrochemical oxidation in the presence of a C₁- to C₆-monoalkyl alcohol.

An anode and cathode made from graphite are used and a yield of 46 percent, of 2,5-dimethoxy-2,5-dihydro-furan was obtained. The selectivity was 51 percent.

It is also possible to use a specific arrangement of electrochemical reactor (cell).

A rectangular electrochemical reactor with a vertical flow can be used to produce the compound of formula (III).

Step (i) comprises the process of the production of a compound of formula (III)

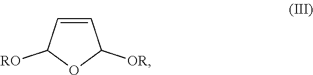

wherein R is a linear or branched C₁-C₆ alkyl group, which comprises electrochemically reacting the compound of formula (II) in Z-form

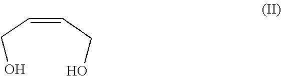

with at least one mono alcohol of formula (IV)

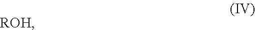

wherein R has the same meaning as in compound of formula (III) characterized in, that
the process is carried out in an electrochemical reactor with a vertical flow.

As it can be seen from formula (II) is in Z-form, when used in the process according to the present invention.

But it is possible that a small amount of the compound of formula (II) in its E-form can be used as well. The E-form can be present in amount of less than 5 wt-%, based on the total weight of the compound of formula (II) in the process.

Preferred compounds of formula (III) are those wherein
R is —CH₃ or —CH₂CH₃.

More preferred is the compound of formula (III) wherein R is —CH₃.

The process of the present invention is usually carried out in non-aqueous medium as a solvent.

In the context of the present invention the term “non-aqueous” means that less than 50 wt-%, based on the total weight of the non-aqueous media, of water can be present in the non-aqueous media.

Usually, the term “non-aqueous” means that less than 20 wt-%, based on the total weight of the non-aqueous media, of water can be present in the non-aqueous media.

The non-aqueous medium comprises usually at least one linear or branched C₁-C₁₀ alcohol as a solvent, preferably at least one linear or branched C₁-C₆ alcohol, more preferably ethanol or methanol, even more preferably methanol. Most preferably, the non-aqueous medium contains one linear or branched C₁-C₆ alcohol, wherein said non-aqueous medium is identical with the mono alcohol of formula (IV) hereinabove.

This means that the mono alcohol of formula (IV) can also serve as non-aqueous medium, or it can be a mixture of other alcohol and the mono alcohol of formula (IV).

It is preferred, that the mono alcohol of formula (IV) is also used as the non-aqueous medium.

The at least one alcohol of formula (IV) is used in an amount of at least 2 mol-equivalents regarding the compound of formula (II). This means that this alcohol is always present in that amount at least, when not used as non-aqueous medium.

Of course, it is also possible that the non-aqueous media is the at least one alcohol of the compound of formula (III).

It is advantageous, that the reaction of step (i) is carried out in an electrochemical reactor with a vertical flow since a better result can be obtained. This means the flow of the reaction mixture can be from bottom to top or from top to bottom of the electrochemical reactor. Preferably the vertical flow of the reaction mixture in the electrochemical reactor is from bottom to top. This is usually done by a pumping system.

Preferably, the flow of the reaction mixture is a circular flow starting from a reservoir going to the electrochemical reactor, and then going back to the reservoir, and wherein more preferably said flow within the electrochemical reactor is a vertical flow from bottom to top.

The size and the form, and therefore also the volume, of the electrochemical reactor can vary. The size and the form, as well as the volume, of the electrochemical reactor is not an essential feature. A very common and also preferred form is a rectangular shape.

The flow rate of the starting material can vary. This depends on the size, form and volume of the rectangular electrochemical reactor.

A usual flow rate is at least 10 mL/min. A usual and preferred range is 10 mL/min to 1000 mL/min.

The electrodes-cathode and electrode-used in the process according to the present invention can be made from any commonly used material only or it can be made from more than one material like a metal on a carrier material or a metal oxide on a carrier material.

Within the process, the target product is formed on the anode and dihydrogen is evolved at the cathode. Therefore, it is advantageously to use a metal or alloy instead of graphite as cathode (graphite is commonly used in the prior art) since they are more active in dihydrogen production. An additional advantage of using a metal or an alloy as cathode is a significantly reduced cell potential, which results in energy savings for the process.

Examples of cathode materials which may be used are metals, graphite, iron, metal alloys, e.g., steel, noble metals, e.g. platinum.

As stated above in a preferred embodiment of the present invention the cathode is not made from graphite.

Materials which are stable under the conditions of the electrolysis are employed for the anode, examples of such materials being noble metals, e.g. platinum, oxides, e.g. ruthenium dioxide on titanium, graphite, highly oriented pyrolytic graphite (HOPG), boron-doped diamond (BDD), dimensionally stable anodes (DSA) and glassy-carbon.

The electrodes can be in any usual form. Such forms can be a plate, wire, a rod, a cell, a mesh, a grid, a sponge, or any other design, which is usually used.

The size of the electrode used in the process according to the present invention can vary and it depends on the size, the form and the structure of the electrochemical reactor (cell).

A usual size is a least 10 cm² (per cell). The upper limit of the electrode is not so critical. Usually, but not necessarily, the cathode and the anode do have the same size.

The reaction medium usually and preferably comprises at least one electrolyte. That can be added to the reaction medium in the form of a salt and/or in form of an acid. Any commonly known and commonly used electrolyte can be used with the exception of phosphoric acid and/or any salt, thereof.

Suitable supporting electrolytes are i.e. HCl, H₂SO₄, Na₂SO₄, NaCl, sodium dodecyl sulfate, methyltributylammonium methylsulfate, triethylammonium bisulfate, tetrabutylammonium bisulfate, tetramethylammonium bisulfate, tetrabutylammonium acetate (NBu₄OAc), tetrabutylammonium sulfate, tetrabutylammonium hexafluorophosphate, tetrabutylammonium tetrafluoroborate, methanesulfonic acid, ammonium bisulfate, tetrabutylphosphonium methanesulfonate, 1-methylimidazolium bisulfate, tetrabutylammonium perchlorate and LiClO₄.

Usually, a concentration of up to 2 M of the at least one electrolyte is used, preferably 0.01-1 M, more preferably 0.1 to 0.5 M, 02.-0.5M.

In preferred embodiment, the electrolyte is not phosphoric acid and/or a salt, thereof.

The pH value of the reaction medium of the process according to the present invention at the start of the process is preferably between 0 and 7.

The process according to the present invention is carried out at a temperature range of 0° C. to 75° C. (preferably 10° C. to 60° C., more preferably, 15° C. to 40° C.).

The process according to the present invention is usually carried out at ambient pressure.

Depending on the cell, the process according to the present invention can be carried out batchwise or in a continuous way. The continuous process is preferred.

The current density used in the process according to the present invention is preferably between 1-1000 mA/cm². Preferably, 10-1000 mA/cm², more preferably 20-1000 mA/cm².

The electrical potential between the anode and cathode may be 12 V or less. A suitable range is 0.5-12 V, preferred is 0.5-10 V; more preferred is 0.5-8 V; most preferred is 1-8 V.

The process according to the present invention can be carried out in galvanostatic or potentiostatic mode.

The reaction product can be isolated using commonly known methods.

An essential feature of the new process according to the present invention is the recycling of the non-reacted alcohol of formula (IV), the recycling to the electrolyte and the removal of the water from the reaction mixture of step (i).

Firstly, the non-reacted alcohol of formula (IV) and the electrolyte are removed using nanofiltration and then the so treated reaction mixture undergoes a pervaporation to remove the water from the reaction before step (ii) of the present process is carried out.

To recycle the alcohol and the electrolyte from the reaction mixture usually a nanofiltration is used. This can be a multistage nanofiltration.

Such a nanofiltration is usually and preferably carried out by using a membrane.

The permeate passes easily through the membrane whereas the retentate is not, or at least significantly less, able to pass the membrane. The higher molecular species are part of the retentate whereas the lower molecular species are part of the permeate.

There are various membranes available which have respective molecular weight cut-off (MWCO) properties. The membrane material can be inorganic or organic.

It is of course substantial that the membrane needs to be stable against the solvents in use as well as resistant to the chosen reaction conditions during the nanofiltration process. However, it has been shown that particularly suitable membranes for the nanofiltration are polymeric membranes, preferably membranes based on silicone, most preferably membranes which are silicone-based composites. Most preferred are membranes based on PDMS/PAN (polydimethylsiloxane/polyacrylonitrile).

As the MWCO is an important property of the membrane used for the separation of the lower and higher molecular species it is preferred for the present invention to use a membrane which has a MWCO of between 100 and 1000 Dalton, preferably of between 150 and 650 Dalton.

Suitable membranes for nanofiltration are commercially available from a variety of suppliers. For example, from Evonik under the trade name PURAMEM® as well as from Borsig GmbH, Solsep BV, UNISOL Membrane Technology or from Inopor.

It is preferred that the nanofiltration is carried out at an elevated pressure, usually and preferably the pressure applied is between 1 and 60 bar, preferably between 2 and 60 bar, more preferably 5 and 50 bar, even more preferably between 10 and 50 bar, most preferably between 15 and 50 bar.

It is preferred that the nanofiltration is carried out at an elevated temperature of between 15 and 70° C., preferably between 15 and 60° C., more preferably between 2° and 50° C.

The equipment used for the nanofiltration is known to the person skilled in the art.

It has been shown that the organic solvent nanofiltration is particularly preferred to be performed in a cross-flow mode.

In the cross-flow mode the feed flows tangentially over the membrane surface and perpendicularly to the permeate flux.

In a particularly preferred manner, the filtrations are performed in a multi-stage nanofiltration. This means that more than one nanofiltration is carried out.

The so (nano)filtrated reaction mixture is further treated to remove the water. This is usually and preferably carried out by pervaporation.

For the pervaporation usually a specific membrane is used.

The membrane needs to be stable against the solvents in use as well as resistant to the chosen reaction conditions during the pervaporation process.

However, it has been shown that particularly suitable membranes for the pervaporation are polymeric membranes, preferably membranes based on polyvinyl alcohols.

Suitable membranes for pervaporation are commercially available from a variety of suppliers (for example from Deltamem or Pervatech).

It is preferred that the pervaporation is carried out at an elevated pressure, usually and preferably the pressure applied is between 0.1 and 30 bar, preferably between 0.5 and 20 bar, more preferably 1 and 30 bar, most preferably a pressure of 1-20 bar is applied on the retentate site and 0.1-10 mbar absolute at the permeate site.

It is preferred that the pervaporation is carried out at an elevated temperature of between 15 and 90° C., preferably between 15 and 80° C., more preferably between 3° and 80° C.

The equipment used for pervaporation is known to the person skilled in the art.

The so treated reaction mixture is then used in step (ii) to produce the compound of formula (I).

### Step (ii)

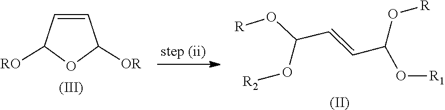

The reaction process of step (ii) is known from the prior art, for example from WO2021/170864A1, WO 2006/108664 and EP 581 097.

Therefore, the process of step (ii) can be carried out according to any of the process known from the prior art (especially from WO2021/170864A1, WO 2006/108664 and EP 581 097).

Preferred are the reaction conditions known from WO2021/170864A1.

The reaction of step (ii) can be carried out in the presence of a catalyst and an alcohol of formula (Va) and/or (Vb)

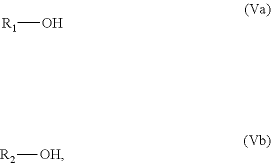

wherein
R₁ is a linear or branched C₁-C₆ alkyl group, and
R₂ is a linear or branched C₁-C₆ alkyl group.

Preferably R₁ is CH₃ or —CH₂CH₃.

Preferably R₁ is CH₃ or —CH₂CH₃.

More preferably R₁ is CH₃.

More preferably R₂ is CH₃ or —CH₂CH₃.

This means that a mixture of alcohols can be used or a pure alcohol, wherein R₁ and R₂ are the same moiety.

Suitable catalysts can be acidic ion exchangers. Such anionic ion exchangers usually have a concentration of acid sites of at least 2.5 eq/kg, preferably at least 3.0 eq/kg, more preferably at least 4.0 eq/kg, most preferably 5.0 eq/kg.

In a more preferred embodiment, the catalyst is an acidic ion exchanger containing sulfonic acid groups and having a concentration of acid sites of at least 2.5 eq/kg, preferably at least 3.0 eq/kg, more preferably at least 4.0 eq/kg, most preferably 5.0 eq/kg.

Such preferred catalysts are either acidic ion exchangers having a particle size distribution greater than or equal to 400 micro m and a water retention capacity <60 percent, preferably a water retention capacity in the range of from 40 to 60 percent, more preferably a water retention capacity in the range of from 50 to 60 percent, or acidic ion exchangers having a particle size distribution <400 micro m and a water retention capacity >60 percent, preferably a water retention capacity in the range of from 60 to 80 percent, more preferably a water retention capacity in the range of from 60 to 75 percent.

The molar ratio of the compound of formula (III) to alcohol of formula (Va) and of formula (Vb) is preferably in the range of from 1:45 to 1:100, more preferably in the range of from 1:50 to 1:90, even more preferably in the range of from 1:60 to 1:80, most preferably in the range of from 1:70 to 1:80.

The obtained compound of formula (I) can be isolated and if desired further purified.

As stated above, the compounds of formula (I) such as 1,1,4,4-tetramethoxy-2-butene, wherein R, R₁ and R₂ are all —CH₃ are important intermediates in the synthesis of carotenoids (known from prior art, i.e., from CN 108 752 178 A).

The following examples serve to illustrate the invention. If not otherwise stated all parts are given are related to the weight and the temperature is given in° C.

### EXAMPLE

Step (i) (Electrolysis of the compound of formula (II) R═CH₃)

Electrochemical oxidation reaction of 1 M Z-2-butene-1,4-diol (2.96 mol) to 2,5-dihydro-2,5-dimethoxyfuran (DMDF) was carried out in an undivided flow-cell (V=10 mL, surface area 100 cm², 1 mm electrode distance) in methanol, 5.5 wt % triethylamine and 4.1 wt % sulfuric acid was used as electrolyte. Graphite (100 cm²) electrode was used as anode and stainless steel (100 cm²) was used as cathode. Electrolysis was carried out galvanostatically by applying a current density of 150 mA/cm² at 20° C. (measured cell potential 6.4 V). The reaction mixture was pumped with a flowrate of 20 L/h through the flow-cell. After 1440 min a conversion of 90%-2-butene-1,4-diol (BED) was achieved, whereas the selectivity of DMDF was 70% at a 56% Faraday efficiency.

Nanofiltration after Step (i))

The reaction mixture obtained in step (i) has been objected to a two-stage nanofiltration. In both organic solvent nanofiltration units the membrane Evonik PuraMem Performance (Evonik Industries AG, Germany) having a surface area of 42 cm² has been used and a pressure of bar applied at 25° C.

After application of 1 Diavolume (DV) of methanol with the nanofiltration of the starting solution the composition of the permeate 1 have been analysed and indicated in table 1. The permeate has been used as starting solution for the second organic solvent nanofiltration unit using the same setup. After application of 1.3 Diavolume (DV) of methanol with the nanofiltration the composition of the permeate 2 of the second organic solvent nanofiltration unit have been analysed and indicated in table 1. As a measure for the overall separation performance the ratio the of for DMDF and electrolyte is given for the starting solution (Electrolysis reaction mixture, introduced into the first organic solvent nanofiltration unit) and the permeate of the first and second organic solvent nanofiltration unit, indicated in table 1.

The comparison of the results shown in table 1 show that particular the OSN membrane Evonik PuraMem Performance shows an extraordinarily good separation performance. Across two stages of OSN, 99.3% of used electrolyte were recovered and separated from DMDF.

TABLE 1

| | C₄ | | | | |
|---|---|---|---|---|---|
| | diacetal | DMDF | BED | | |
| | [w/w %, | [w/w %, | [w/w %, | Electrolyte | Ratio |
| | q-NMR] | q-NMR] | q-NMR] | [w/w %, q-NMR] | DMDF/Electrolyte |
| Feed (reaction | 0.5 | 8.8 | 0.4 | 6.1 | 1.4 |
| mixture after | | | | | |
| step (i) | | | | | |
| After first | 0.2 | 6.5 | 0.2 | 0.4 | 15.4 |
| nanofiltration | | | | | |
| After second | 0.1 | 4.3 | 0.1 | 0.04 | 106.5 |
| nanofiltration | | | | | |

Pervaporation after the Nanofiltrations

The permeate OSN obtained after the nanofiltrations has been objected to a pervaporation. In the pervaporation unit the membrane DeltaMem PERVAP 4101 (DeltaMem AG) having a surface area of 170 cm² has been used at 70° C. and a pressure of 15 bar applied on the retentate site and 1 mbar absolute at the permeate site.

After 74 h of pervaporation the composition of the retentate have been analysed and indicated in table 2 (MeOH wash means additional equipment wash, solution unified). As a measure for the overall performance, the water content is given for the starting solution (permeate OSN stage 2) and the retentate, indicated in table 2.

The comparison of the results shown in table 2 show that the water content of the DMDF-containing solution was significantly reduced.

TABLE 2
results of the pervaporation step

| | C₄-diacetal | DMDF | Water | Total weight |
|---|---|---|---|---|
| | [g, q-NMR] | [g, q-NMR] | [g, q-NMR] | [g] |
| Feed (reaction | 1.01 | 27.9 | 5.3 | 465 |
| mixture | | | | |
| after the | | | | |
| nanofiltrations) | | | | |
| Retentate | 0.91 | 24.4 | 1.7 | 480 |
| (+MeOH wash) | | | | |

Step (ii) (Acid-Catalyzed Conversion of DMDF to Compound of Formula (I) with R, R₁, R₂═CH₃)

The reactor is filled with 50 mm glass wool and the acidic ion exchanger DOWEX50WX4 (=catalyst) is added. Then methanol is pumped through the catalyst bed until the solvent is colorless. Various reaction mixture compositions in methanol were pumped through the catalyst bed at 21° C. with a specific residence time given in the table 3. Samples are taken after certain periods. The starting solution as well as the samples are analyzed by qNMR. The results in below table 3 clearly show the need of the downstream processing steps from the electrolysis mixture before the reaction mixture (=mainly DMDF in methanol) is further reacted within step (ii).

TABLE 3

| | Water | Residence | Content DMDF | Conversion | Selectivity | Space-time |
|---|---|---|---|---|---|---|
| | content | time | in MeOH | of DMDF | C₄-diacetal | Yield |
| Reaction Mixture | [wt %] | [min] | [wt %] | [%] | [%] | [g/hL] |
| neat DMDF | — | 17 | 4.7 | 46.3 | 89.0 | 72.0 |
| Electrolysis Reaction mixture | 2.0 | 17 | 3.0 | 5.0 | 79.0 | 5.0 |
| Permeate OSN stage 2 | 1.70 | 17 | 4.3 | 24.3 | 80.4 | 34.5 |
| Pervaporation Retentate | 0.36 | 35 | 4.6 | 40.1 | 96.7 | 33.6 |
| neat DMDF | 0.40 | 35 | 4.7 | 48.7 | 89.1 | 38.0 |
| Pervaporation Retentate | 0.18 | 35 | 4.6 | 50.0 | 90.2 | 38.7 |
| neat DMDF | 0.20 | 35 | 4.7 | 55.0 | 91.4 | 43.6 |

1. Process for the production of the compound of formula (I) 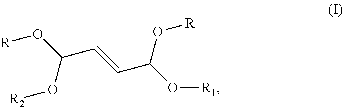
wherein R, R₁ and R₂ are independently from each other linear or branched C₁-C₆ alkyl group,
wherein
in a first step (step (i)) a compound of formula (II) in Z-form 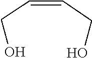
is reacted electrochemically
with at least one mono alcohol of formula (IV) 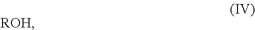
wherein R has the same meaning as in compound of formula (I) and then
in a second step (step (ii)), the reaction product of step (i), which is the compound of formula (III) 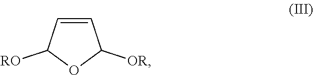
wherein R has the same meaning as in compound of formula (I) is reacted in the presence of a catalyst and at least one alcohol of formula (Va) and/or (Vb) 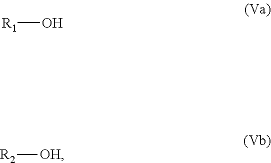
wherein
R₁ is a linear or branched C₁-C₆ alkyl group, and
R₂ is a linear or branched C₁-C₆ alkyl group,
characterized in that after step (i) and before step (ii)
the reaction mixture is undergoing at least one nanofiltration step followed by at least one pervaporation step.

2. Process according to claim 1, wherein
R, is —CH₃ or —CH₂CH₃, and
R₁ is —CH₃ or —CH₂CH₃, and
R₂ is —CH₃ or —CH₂CH₃.

3. Process according to claim 1, wherein
R, R₁ and R₂ are —CH₃.

4. Process according to claim 1, wherein the reaction of step (i) is carried out in a non-aqueous medium, wherein in such medium less than 50 wt-%, based on the total weight of the non-aqueous media, of water can be present.

5. Process according to claim 1, wherein the reaction of step (i) is carried out in at least one linear or branched C₁-C₁₀ alcohol as a solvent, preferably in at least one linear or branched C₁-C₆ alcohol.

6. Process according to claim 1, wherein the flow rate in step (i) is 10 mL/min to 1000 mL/min.

7. Process according to claim 1, wherein the electrochemical process of step (i) is carried out in the presence of at least one electrolyte chosen from the group consisting of HCl, H₂SO₄, Na₂SO₄, NaCl, sodium dodecyl sulfate, methyltributylammonium methylsulfate, triethylammonium bisulfate, tetrabutylammonium bisulfate, tetramethylammonium bisulfate, tetrabutylammonium acetate (NBu₄OAc), tetrabutylammonium sulfate, tetrabutylammonium hexafluorophosphate, tetrabutylammonium tetrafluoroborate, methanesulfonic acid, ammonium bisulfate, tetrabutylphosphonium methanesulfonate, 1-methylimidazolium bisulfate, tetrabutylammonium perchlorate and LiClO₄.

8. Process according to claim 1, wherein the at least one nanofiltration is carried out by using a polymeric membrane.

9. Process according to claim 8, wherein the membrane has a molecular weight cut-off of between 100 and 1000 Dalton.

10. Process according to claim 1, wherein the at least one nanofiltration is carried out at a pressure between 1 and 60 bar.

11. Process according to claim 1, wherein the at least one nanofiltration is carried out at a temperature of between 15 and 70.

12. Process according to claim 1 wherein the at least one pervaporation is carried out by using a polymeric membrane, preferably a membrane based on polyvinyl alcohols.

13. Process according to claim 1, wherein the pervaporation is carried out at a pressure of 1-20 bar.

14. Process according to claim 1, wherein the alcohol used is either a mixture of alcohols, or a pure alcohol wherein R₁ and R₂ are the same moiety, preferably wherein the alcohol is a pure alcohol wherein R₁ and R₂ are the same moiety.

15. Process according to claim 1, wherein the catalyst of step (ii) is an acidic ion exchanger.
